(19) United States
(12) Patent Application Publication
(10) Pub. No.: US 2023/0263643 A1
(43) Pub. Date: Aug. 24, 2023

(54) Antimicrobial Prosthetic Liner

(71) Applicants: Aldo Laghi, Pinellas Park, FL (US); Nathaniel Vint, Palm Harbor, FL (US)

(72) Inventors: Aldo Laghi, Pinellas Park, FL (US); Nathaniel Vint, Palm Harbor, FL (US)

(21) Appl. No.: 18/112,683

(22) Filed: Feb. 22, 2023

Related U.S. Application Data
(60) Provisional application No. 63/313,563, filed on Feb. 24, 2022.

(51) Int. Cl. A61F 2/50; A01N 59/16

(52) U.S. Cl. CPC A61F 2/50; A01N 59/16

(57) ABSTRACT

An antimicrobial prosthetic liner having an exterior fabric layer and an inner layer comprising a thermoplastic and further incorporating evenly distributed nanoparticles of certain metal oxide nanoparticles in an amount ranging from 2%-6% weight per weight and ranging in size from between 5 nanometers and 100 nanometers in diameter, most preferably 20 nanometers in diameter. Preferably, the metal oxide nanoparticles used are titanium dioxide and zinc oxide. Copper oxide, magnesium oxide, aluminum hydroxide as well as metal nanoparticles such as gold nanoparticles and silver nanoparticles may also be used to provide antimicrobial effects.

### CROSS-REFERENCE TO RELATED APPLICATIONS

This applications claims priority to pending provisional application No. 63/313,563 filed Feb. 24, 2022, the disclosure of which is incorporated by reference herein.

### BACKGROUND OF THE INVENTION

### Field of the Invention

The described invention relates to liners for use in a prosthetic assembly. Specifically, the described invention relates to liners incorporating metal nanoparticles and/or metal oxide nanoparticles which have been shown to have antimicrobial properties.

### Description of the Background Art

Good hygiene is important for the health of residual limbs. Most lower limb amputees are of vascular origin as opposed to traumatic amputees, they tend to be elderly and, possibly, with limited hand dexterity and stamina. Ideally a prosthetic liner is washed with soap daily, as is the residual limb, in order to avoid the growth of microorganisms. Limited dexterity or stamina may limit the efficacy or required duration of this washing. Dangerous microorganisms such a Staphylococcus aureus, Escherichia coli and Candida albicans often colonize the skin of most people, without adverse consequences. However, if they enter the bloodstream in sufficient quantity and the host has an impaired lymphatic system, such as in diabetic patients, they can cause serious infections, which, in turn can lead to additional amputation or sepsis and even death.

Prosthetic socks containing antimicrobial agents are currently available in the market. Examples include the comfort silver sheath which contains silver knitted into the sheath preventing microbial growth. Another example is the liner wand which is an applied coating of silver carboxylate that is placed directly onto a liner surface but must be maintained. Amputees in most industrialized countries, however, utilize prosthetic liners for comfort and suspension. A prosthetic liner is worn in contact with the skin while prosthetic socks are generally used for volume fluctuation of the residual limb and are worn outside of the liner. The prosthetic liner, in order to provide the suction required to suspend the prosthesis, is a closed system, which insulates the residual limb and promotes sweating which, in turn, promotes fungal growth.

Prosthetic liners come in three forms: silicone, styrene triblock copolymer gels, and urethane. The styrene triblock copolymer gels most commonly used in prosthetic liners are SEBS, SEEPS, and SEPS. SEBS plastics are styrene-ethylene-butylene-styrene chain copolymers. This chain behaves like rubber without undergoing vulcanization. SEBS plastics are strong and flexible while having excellent heat and UV resistance. SEEPS plastics are styrene-[ethylene-(ethylene-propylene)]-styrene styrenic block copolymer. SEEPS based thermoplastic elastomers can be used for many applications with many functions as a matrix, compatibilizer (i.e. allowing for multiple materials to work together as a cohesive whole), modifier or adhesive. SEEPS has good resistance to oxidizing agents, weathering, aging, and it can be used under various conditions. SEPS plastics are styrene-ethylene-propylene-styrene, block copolymers. SEPS based thermoplastic elastomers are very flexible, have excellent heat and UV resistance, and are easy to process. It is produced by partial and selective hydrogenating of styrene-isoprene-styrene copolymers (SIS) which improves the thermal stability, weathering and oil resistance, and makes SEPS steam sterilisable. However, hydrogenation also reduces the mechanical performance and increases the cost of the polymer.

Metals and metal oxides have been long known to provide strong bactericidal action, but many are not suitable for prolonged skin contact as they can be leached by sweat into the skin causing allergic or even toxicological reactions. Metal and metal oxide nanoparticles have been tested with regard to antimicrobial activity and have been found to be efficient in the removal of pathogens. While metal and metal oxide nanoparticles may not show considerable activity in the form of a metal oxide or metal salt alone due to their tendency to aggregate, their stability and slow release of metal ions when in nanoparticle form create the possibility of synthesis with pathogen prevention. As seen in FIGS. 1 and 2, studies have shown a drastic decrease of Escherichia Coli (E. coli) and Staphylococcus aureus populations when exposed to oxide and hydroxide-loaded thermoplastic elastomer compounds in various concentrations.

Indeed, as shown in FIGS. 3 and 4, titanium dioxide is extremely effective at reducing Candida albicans, a bacteria which causes candidiasis (i.e. a fungal infection). In FIG. 4, each group corresponds to 2%, 4%, 6%, 8%, and 10% w/w titanium dioxide in order. It is also known in the art that introduction of metal oxides to thermoplastic elastomers does not change the elastomer's tensile properties, as shown in FIG. 5.

Therefore, it is an object of this invention to provide an improvement which overcomes the aforementioned inadequacies of the prior art devices and provides an improvement which is a significant contribution to the advancement of the prosthetic liner art.

Another object of the invention is to provide a prosthetic liner with antimicrobial properties.

Another object of the invention is to provide a prosthetic liner that does not promote fungal growth.

Another object of the invention is to provide a prosthetic liner that incorporates metal oxide nanoparticles.

Another object of the invention is to provide a prosthetic liner that incorporates metal nanoparticles.

Another object of the invention is to provide a prosthetic liner that incorporates a mixture of metal nanoparticles and metal oxide nanoparticles.

Another object of the invention is to provide a prosthetic liner that incorporates a mixture of metal oxide nanoparticles.

The foregoing has outlined some of the pertinent objects of the invention. These objects should be construed to be merely illustrative of some of the more prominent features and applications of the intended invention. Many other beneficial results can be attained by applying the disclosed invention in a different manner or modifying the invention within the scope of the disclosure. Accordingly, other objects and a fuller understanding of the invention may be had by referring to the summary of the invention and the detailed description of the preferred embodiment in addition to the scope of the invention defined by the claims taken in conjunction with the accompanying drawings.

### SUMMARY OF THE INVENTION

The present invention relates generally to an antimicrobial prosthetic liner having an exterior fabric layer and an inner layer comprising an elastomer and further incorporating evenly distributed nanoparticles of certain metal or metal oxide nanoparticles in an amount ranging from 2%-6% weight per weight (w/w) and ranging in size from between 5 nanometers and 100 nanometers, most preferably 20 nanometers in diameter. Preferably, the metal oxide nanoparticles used are titanium dioxide and zinc oxide. Copper oxide, magnesium oxide, aluminum hydroxide, as well as metal nanoparticles such as gold nanoparticles and silver nanoparticles may also be used to provide antimicrobial effects.

Incorporation of the metal and or metal oxide particles is dependent upon the even distribution throughout the body of the material. This is achieved by first a preparation of the nanoparticles in a suitable carrier for the material. Utilizing silicone oil allows for the dispersal of a given percentage of the nanoparticles within a silicone elastomer. This allows the nanoparticles to be further distributed through the elastomer material in a further step but ensuring that the particles are dispersed and not clumping which would reduce their efficiency. This distribution in a carrier material can be done through but not limited to high shear mixing, impacts through a ball mill, and vacuum dispersion although any method that allows for even distribution may work.

The foregoing has outlined rather broadly the more pertinent and important features of the present invention in order that the detailed description of the invention that follows may be better understood so that the present contribution to the art can be more fully appreciated. Additional features of the invention will be described hereinafter which form the subject of the claims of the invention. It should be appreciated by those skilled in the art that the conception and the specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a more complete understanding of the present disclosure and its advantages, reference is now made to the following descriptions, taken in conjunction with the accompanying drawings, in which:

FIG. 1 is a prior art graph showing the bactericidal properties of various metal oxides against Escherichia coli;

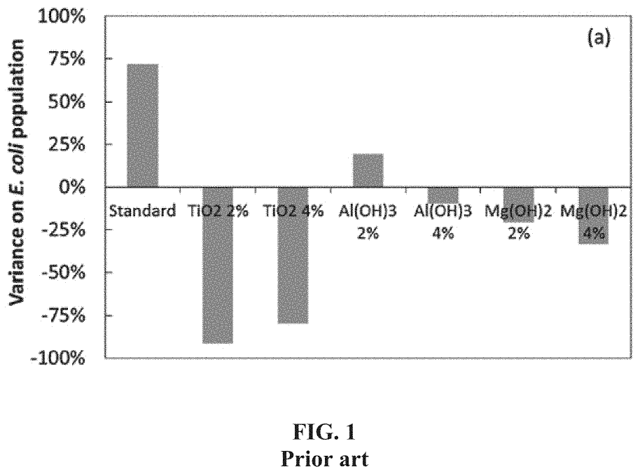

FIG. 2 is a prior art graph showing the bactericidal properties of various metal oxides against Staphylococcus aureus;

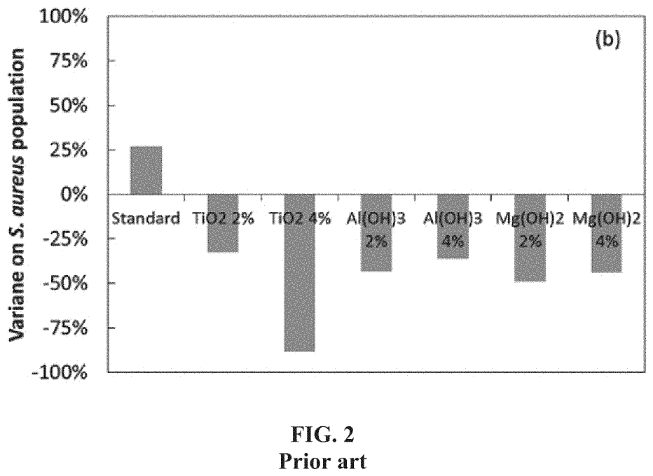

FIG. 3 is a prior art graph showing the bactericidal properties of titanium dioxide against Candida albicans;

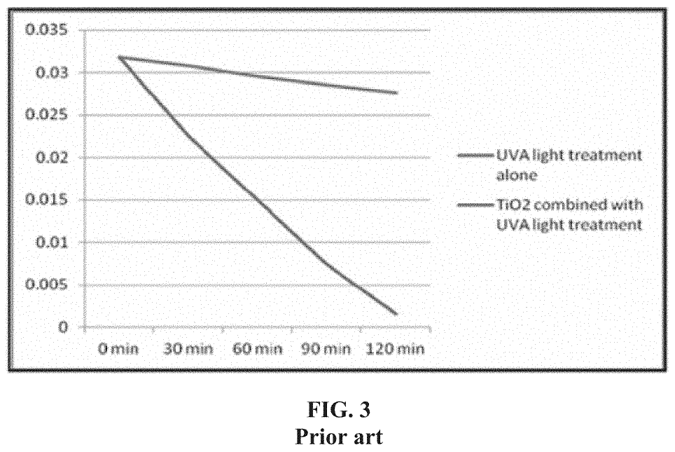

FIG. 4 is a prior art table showing reduction in Candida albicans over time after on silicone elastomer containing various percentages of titanium dioxide;

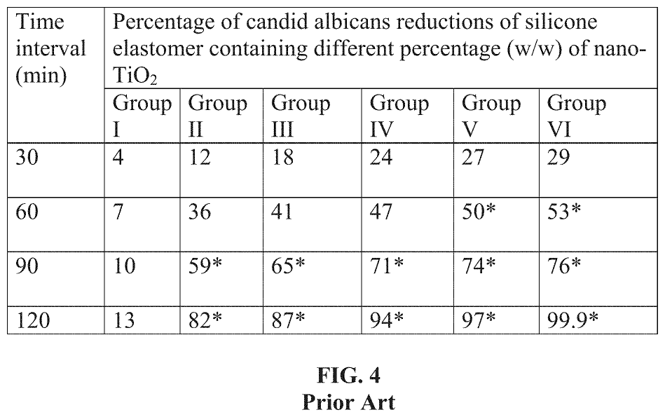

FIG. 5 is a prior art table providing various mechanical properties of thermoplastic elastomers containing various metal oxides;

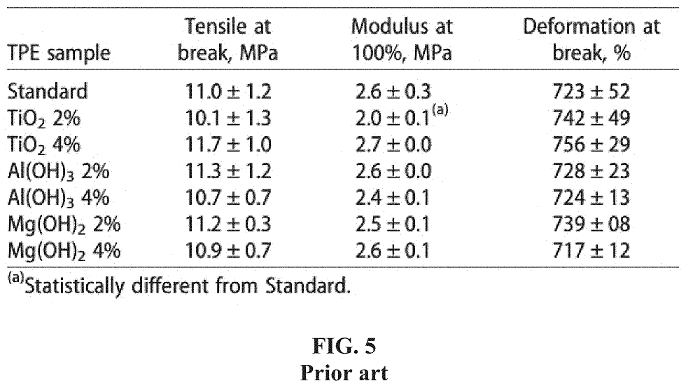

FIG. 6 is a front perspective view of the antimicrobial prosthetic liner; and

FIG. 7 is a cross-sectional view of the antimicrobial prosthetic liner.

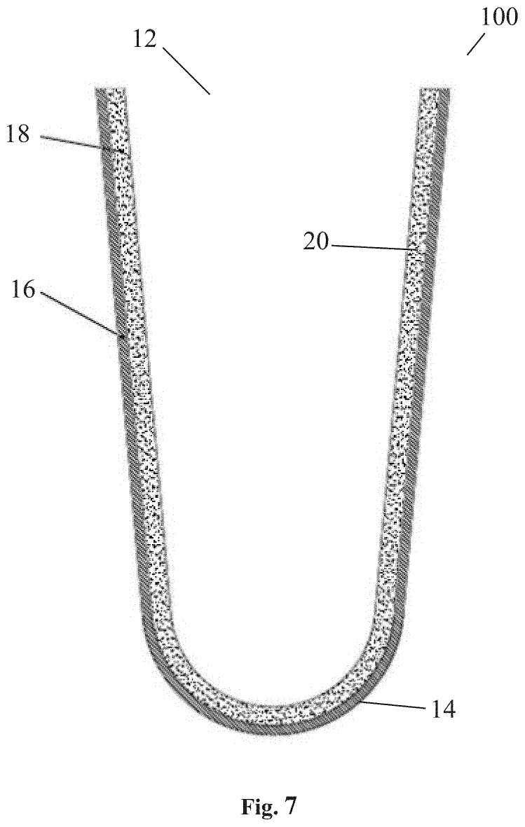

Similar reference numerals refer to similar parts throughout the several views of the drawings.

### DETAILED DESCRIPTION OF THE PREFERRED EMBODIMENT

The following description is of the best mode presently contemplated for carrying out the invention. This description is not to be taken in a limiting sense, but is made merely for the purpose of describing one or more preferred embodiments of the invention. The scope of the invention should be determined with reference to the claims.

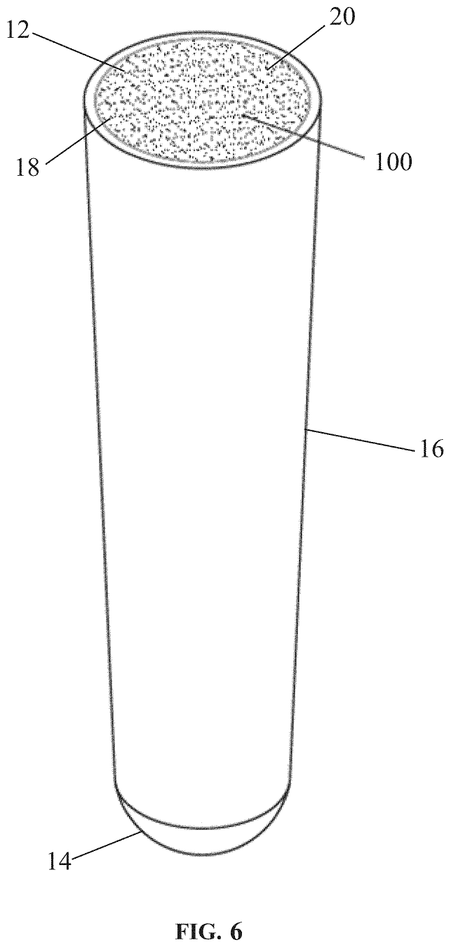

The present invention relates to a liner 100 for use with prosthetic devices. As shown in FIG. 6, the liner 100 for use with a prosthetic assembly comprises an open upper end 12 for receiving a residual limb, not shown, a closed distal end 14, and sidewalls 16 of predetermined thickness. The liner is airtight when donned over a residual limb. The preferred thickness of the sidewalls 16 is between 1-9 millimeters. The sidewalls 16 have an inner layer 18 of thermoplastic which may be silicone, styrene triblock copolymer gels, or urethane. The sidewalls 16 can be fabric or another layer of more durable and higher friction silicone.

The inner layer 18 is preferably infused with metal oxide particles 20 distributed evenly throughout its mass. This metal oxide is chosen from the group comprising of: silver nanoparticles, copper(I) oxide, copper(II) oxide, magnesium oxide, titanium dioxide, and zinc oxide. This list is not limiting and should be construed to encompass nanoparticles of metals and metal oxides suitable for antimicrobial liners now known or to be developed.

The metal oxides used in the present invention are used because they cause oxidative stress on the cell wall of the bacteria and other microbes that form over time when using prosthetic liners. Silver nanoparticles have bactericidal properties due to disassociation of silver ions and have long been used in treatment of infections, wounds, and burns. Silver nanoparticles having a diameter of less than 20 nanometers gain their bactericidal properties by attaching to sulfur-containing proteins in bacterial cell membranes which then induces a change in the membrane's permeability causing pores in the cell walls which then allow silver ions to infiltrate the cells causing damage as it attempts to pump the silver nanoparticles out eventually causing destruction of the cell membrane. Copper(I) oxide and copper(II) both have potent antibacterial activity due to reactive oxygen species (ROS) production after they attach to the bacterial cell walls which then causes oxidative stress and eventually destruction. Magnesium oxide may be more attractive than some of the other metal oxide options because it can be degraded and metabolized efficiently in the human body so long as renal function in the subject is normal. Titanium oxide demonstrates excellent antifungal and antibacterial properties against a broad range of Gram-positive and Gram-negative bacteria and is chemically stable, non-toxic, inexpensive, and is considered a Generally Recognized as Safe (GRAS) substance. Titanium oxide is also photocatalytic which means that the bactericidal properties of titanium oxide are accelerated in the presence of light energy by participating in redox reactions (which eventually destroy the bacterial cell wall) through the creation of electron donor and electron acceptor particles.

Zinc oxide is of particular importance for the present invention as it is a bio-safe material that possesses photo-oxidizing and photocatalytic properties. Zinc oxide has been shown to generate ROS including hydrogen peroxide, hydroxyl radicals, and peroxide. As described above, ROS is a major factor in mechanisms for cell wall damage in bacteria and, in this case, specifically with zinc oxide-localized interactions creating enhanced membrane permeability, loss of proton motive force, and uptake of dissolved zinc ions toxic to the cell. These mechanisms lead to mitochondria weakness, intracellular outflow, and release in gene expression of oxidative stress which causes eventual cell growth inhibition and cell death. In some cases, enhanced antibacterial activity can be attributed to surface defects due to the abrasive cell surface texture created by zinc oxide. Zinc oxide is non-toxic to and biocompatible with human cells.

The present invention infuses these metal oxide particles 20 into the inner layer 18 of the prosthetic liner 100, with the metal oxide particles 20 having a size range from 5 nanometers to 100 nanometers in diameter, preferably between 10 nanometers and 40 nanometers. The most preferred nanoparticles diameter is 20 nanometers. The amount of metal oxide particles 20 present in the inner layer 18 should range from 2% to 6% w/w and may be a single metal or metal oxide nanoparticle or a blend of those described herein.

In addition, other formulations that do not incorporate metal oxides may also be effective. For example, triclosan, chlorinated diphenyl ether, and isothiazolinone are all effective antifungal and antibacterial organic chemicals that could be incorporated into the thermoplastic inner layer 18. These organic chemicals may be included as a blend or present individually but overall should be present in a range between 0.0001% to 3% w/w in relation to the thermoplastic inner layer.

The present disclosure includes that contained in the appended claims, as well as that of the foregoing description. Although this invention has been described in its preferred form with a certain degree of particularity, it is understood that the present disclosure of the preferred form has been made only by way of example and that numerous changes in the details of construction and the combination and arrangement of parts may be resorted to without departing from the spirit and scope of the invention.

Now that the invention has been described,

What is claimed is:

1. An antimicrobial prosthetic liner comprising:
an open upper end;
a closed distal end; and
sidewalls between the closed distal end and the open upper end; and
an inner layer of a thermoplastic material wherein the thermoplastic material is infused with metal oxide particles which are distributed evenly throughout the thermoplastic material in an amount between 2-10% by weight.

2. The antimicrobial prosthetic liner of claim 1 wherein the metal oxide is selected from the group consisting of silver nanoparticles, copper(I) oxide, copper(II) oxide, magnesium oxide, titanium dioxide, and zinc oxide.

3. The antimicrobial prosthetic liner of claim 2 wherein the metal oxide into the thermoplastic material is a mixture of metal oxide particles.

4. The antimicrobial prosthetic liner of claim 1 wherein the sidewalls have a thickness between 1-9 millimeters.

5. The antimicrobial prosthetic liner of claim 1 wherein the metal oxide particles have a diameter between 5-100 nanometers.

6. An antimicrobial prosthetic liner comprising:
an open upper end;
a closed distal end; and
sidewalls between the closed distal end and the open upper end and having a thickness between 1-9 millimeters; and
an inner layer of a thermoplastic material wherein the thermoplastic material is infused with metal oxide particles having a diameter between 5-100 nanometers selected from the group consisting of silver nanoparticles, copper(I) oxide, copper(II) oxide, magnesium oxide, titanium dioxide, and zinc oxide which are distributed evenly throughout the thermoplastic material in an amount between 2-10% by weight.

7. An antimicrobial prosthetic liner comprising:
an open upper end;
a closed distal end; and
sidewalls between the closed distal end and the open upper end; and
an inner layer of a thermoplastic material wherein the thermoplastic material is infused with metal oxide particles in an amount effective to cause oxidative stress on bacteria when donned on a residual limb.

8. The antimicrobial prosthetic liner of claim 7 wherein the metal oxide is selected from the group consisting of silver nanoparticles, copper(I) oxide, copper(II) oxide, magnesium oxide, titanium dioxide, and zinc oxide.

9. The antimicrobial prosthetic liner of claim 8 wherein the metal oxide into the thermoplastic material is a mixture of metal oxide particles.

10. The antimicrobial prosthetic liner of claim 7 wherein the sidewalls have a thickness between 1-9 millimeters.

11. The antimicrobial prosthetic liner of claim 7 wherein the metal oxide particles have a diameter between 5-100 nanometers.
